(19) United States
(12) Patent Application Publication
(10) Pub. No.: US 2013/0165508 A1
(43) Pub. Date: Jun. 27, 2013

(54) METHODS AND FORMULATIONS FOR TREATING INEFFECTIVE OR DECREASED ESOPHAGAL MOTILITY

(71) Applicant: Robert C. Stover, Lewis Center, OH (US)

(72) Inventor: Robert C. Stover, Lewis Center, OH (US)

(21) Appl. No.: 13/771,334

(22) Filed: Feb. 20, 2013

Related U.S. Application Data
(63) Continuation-in-part of application No. 13/430,266, filed on Mar. 26, 2012, now Pat. No. 8,383,684.
Application No. 13/430,266 is a continuation-in-part of application No. 12/210,995, filed on Sep. 15, 2008, now Pat. No. 8,143,314.
(60) Provisional application No. 61/039,448, filed on Mar. 26, 2008.
(60) Provisional application No. 60/972,123, filed on Sep. 13, 2007.

(51) Int. Cl. A61K 31/325

(52) U.S. Cl. CPC A61K 31/325
USPC 514/478

(57) ABSTRACT

Disclosed embodiments describe pharmaceutical compositions and methods for treating ineffective esophageal motility in which bethanechol and pharmaceutically acceptable absorption enhancers including bile acids and mixtures thereof are topically introduced to the esophagus. Therapeutically effective amounts of bethanechol are delivered while reducing or eliminating parasympathetic nervous system side effects normally associated with systemic bethanechol delivery.

### CROSS-REFERENCE TO RELATED APPLICATIONS

This application is a continuation-in-part of U.S. Ser. No. 13/430,266, filed 26 Mar. 2012, now U.S. Pat. No. 8,383,684, issued 26 Feb. 2013, which is in turn a continuation-in-part of U.S. Pat. No. 12/210,995, filed 15 Sep. 2008, now U.S. Pat. No. 8,143,314, issued 27 Mar. 2012, which is a non-provisional of U.S. 60/972,123, filed Sep. 13, 2007, and U.S. 61/039,448, filed Mar. 26, 2008. Each of the aforementioned applications is incorporated by reference as if fully recited herein and a claim of priority is made to each.

### TECHNICAL FIELD

This description relates generally to the treatment of ineffective or decreased esophageal motility and particularly to methods and formulations comprising bethanechol to enhance the swallowing mechanism.

### BACKGROUND

The mechanism of swallowing, also known as deglutition, while generally not given much consideration by healthy individuals, is highly complex and involves the control and coordination of a number of physiologic processes within the body. Initially, food in the mouth is prepared for entry into the esophagus and eventually the stomach and the rest of the gastrointestinal (GI) tract. In the case of solid food, it is chewed, or masticated, and formed into a bolus using the jaw muscles, the teeth, and the tongue. In the case of liquids, the appropriately sized amount of liquid is prepared. Next, the bolus enters the pharynx and subsequently into the esophagus. Peristalsis moves the bolus from the pharynx, through the esophagus, and into the stomach after passing a lower esophageal sphincter (LES). The swallowing process in the pharynx and the esophagus is coordinated by the autonomic nervous system. Located within the smooth and striated muscle layers of the pharynx and the esophagus are various muscarinic receptors which, although not currently completely understood, when activated, appear to effect peristalsis. These muscarinic receptors help control the smooth and striated muscle layers not only of the esophagus, however, but of the lower gastrointestinal tract, and the bladder. They thus effect peristaltic waves to move materials through the esophagus and the rest of the GI tract and, in the case of the bladder, discharge urine.

Ineffective esophageal motility is associated with many disorders affecting humans. For example, human immunodeficiency virus (HIV) and cancer chemotherapy and radiation patients often have esophageal motility problems related to the disease processes themselves and/or from side effects of treatment. Patients with HIV, in particular, are currently treated with multi-medication regimens often requiring multiple daily doses. Unfortunately, missing doses of medication can lead to medication resistance in those patients, compromising treatment. In addition, the elderly and patients suffering from strokes or traumatic brain or spinal cord injury, Parkinson's Disease, multiple sclerosis, multiple system atrophy with autonomic phenomena (formerly known as Shy-Drager syndrome), and amyotrophic lateral sclerosis often suffer from ineffective esophageal motility. It has been reported that over one-half of all stroke victims suffer from acute ineffective esophageal motility. It has also been reported that the majority of elderly Parkinson's patients die from bronchial pneumonia and infectious shock and choking is one of the main causes of aspiration pneumonia. Finally, certain drugs can cause such an effect. Examples include antimuscarinics (muscarinic receptor antagonists), including, for example, tolterodine.

Bethanechol, available as 2-carbamoyloxpropyl-trimethyl-ammonium chloride, is a parasympathomimetic drug that exerts its effect directly and selectively on the muscarinic M2 receptors and, it is believed, to a certain extent, on the M4 receptors and the M3 receptors and is not inactivated by acetylcholinesterase. See, Liwang L., et at., “Pharmacological discrimination between muscarinic receptor signal transduction cascades with bethanechol chloride.” British Journal of Pharmacology, 138 (2003) 1259-70. Bethanechol has been used for some time for the treatment of urinary retention associated with neurogenic bladder, to stimulate lower gastrointestinal motility, and to help prevent gastroesophageal reflux disease (GERD). Formulations of bethanechol consist of either tablets, sublingual tablets, subcutaneous injection, or an oral solution having, for example, bethanechol chloride (5 mg/ml) combined with a suspending vehicle, such as is provided by products sold under the registered trademarks ORA-PLUS® or ORA-SWEET® (both from Paddock Laboratories, Inc., Minneapolis, Minn.) or by cherry syrup. Recently, in 50 mg oral doses, bethanechol was shown, after 15 and 40 minutes, to improve esophageal motility. See, Agrawal, A., et at., “Bethanechol improves smooth muscle function in patients with severe ineffective esophageal motility.” Journal of Clinical Gastroenterology, 41 (4) (2007) 366-70. All such dosing is designed to be absorbed into the blood stream to be made available to the muscarinic receptors. When provided systemically, however, bethanechol can cause undesirable side effects, including diarrhea, flushing, increased sweating, nausea, stomach pain, or gas, and, importantly, urinary urgency. In the case of HIV and cancer chemotherapy patients, moreover, since nutrient absorption is important in treating these patients, the side effects of diarrhea and abdominal cramping preclude the systemic application of bethanechol for esophageal motility problems in these patients. In fact, there is currently no safe and effective treatment for esophageal motility disorders in these patients. Thus, there exists a need for a formulation and method for effectively treating ineffective esophageal motility while eliminating or reducing undesirable side effects associated with systemic dosing.

As noted above, gastroesophageal reflux disease (GERD) is related to ineffective esophageal motility in that incomplete closure of the lower esophageal sphincter (LES) permits leakage, or reflux, of stomach contents into the esophagus where the acidic nature of the contents will irritate the esophagus. Typical treatment for GERD is not directed at closure of the LES, but is instead directed at neutralizing stomach acids or decreasing the amount of stomach acid produced (the proton pump inhibitors, or PPIs) or released (the H2 blockers). All of these treatments have accompanying side effects and disadvantages.

### SUMMARY

To meet these needs, the present disclosure describes a low-dose bethanechol formulation comprising bethanechol, a pharmaceutically acceptable absorption enhancer such as an ox bile acid mixture, and a pharmaceutically acceptable carrier such as water and a method of treatment for applying the formulation to the pharynx and esophagus. This formulation and method of treatment allows for the topical delivery of a therapeutically effective amount of bethanechol to the muscarinic receptors in the pharynx and esophagus while reducing or eliminating the undesirable side effects associated with traditional bethanechol formulations and methods of delivery for the treatment of ineffective esophageal motility.

In one general aspect, a pharmaceutical composition for the treatment of ineffective esophageal motility is described.

In one embodiment, the composition comprises from about 15 millimolar to about 70 millimolar bethanechol or pharmaceutically acceptable salts thereof; about 500 millimolar to about 700 millimolar one or more pharmaceutically acceptable bile acids; and a pharmaceutically acceptable liquid carrier. In a further embodiment, the concentration of bethanechol is about 25 millimolar. In a further embodiment, the parasympathetic nervous side effects normally associated with the administration of bethanechol are reduced or virtually eliminated.

In a further embodiment, the bile acids comprise taurocholic acid, pharmaceutically acceptable salts thereof, or pharmaceutically acceptable metabolically related derivatives thereof; taurochenodeoxycholic acid, pharmaceutically acceptable salts thereof, or pharmaceutically acceptable metabolically related derivatives thereof; and taurodeoxycholic acid, pharmaceutically acceptable salts thereof, or pharmaceutically acceptable metabolically related derivatives thereof. In a further embodiment, the pharmaceutically acceptable liquid carrier is water.

In a further embodiment, a pharmaceutical composition for the treatment of ineffective esophageal motility is described which composition comprises a pharmaceutically acceptable muscarinic receptor agonist; a pharmaceutically acceptable absorption enhancer; and a pharmaceutically acceptable liquid carrier, wherein the composition is suitable for topical application to the back of the throat, pharynx, or esophagus of a patient in need of treatment; and parasympathetic nervous system side effects normally associated with the muscarinic receptor agonist are reduced or virtually eliminated. In a further embodiment, the muscarinic receptor agonist is selective to M2 receptors. In a further embodiment, the muscarinic receptor agonist is bethanechol.

In a further embodiment, the absorption enhancer comprises a bile acid mix of taurocholic acid, pharmaceutically acceptable salts thereof, or pharmaceutically acceptable metabolically related derivatives thereof; taurochenodeoxycholic acid, pharmaceutically acceptable salts thereof, or pharmaceutically acceptable metabolically related derivatives thereof; and taurodeoxycholic acid, pharmaceutically acceptable salts thereof, or pharmaceutically acceptable metabolically related derivatives thereof. In a further embodiment, the muscarinic receptor agonist is bethanechol and the molar ratio of the bile acid mix to bethanechol is from about 10:1 to about 40:1.

In a further embodiment, the absorption enhancer is chosen from the group consisting of: taurocholic acid, pharmaceutically acceptable salts thereof, or pharmaceutically acceptable metabolically related derivatives thereof; glycocholic acid, pharmaceutically acceptable salts thereof, or pharmaceutically acceptable metabolically related derivatives thereof; glychocholic acid, pharmaceutically acceptable salts thereof, or pharmaceutically acceptable metabolically related derivatives thereof; taurochenodeoxycholic acid, pharmaceutically acceptable salts, or pharmaceutically acceptable metabolically related derivatives thereof; taurodeoxycholic acid, pharmaceutically acceptable salts thereof, or pharmaceutically acceptable metabolically related derivatives thereof; glycochenodeoxycholic acid, pharmaceutically acceptable salts thereof, or pharmaceutically acceptable metabolically related derivatives thereof; ox bile extract; propylene glycol; cholic acid, pharmaceutically acceptable salts thereof, pharmaceutically acceptable metabolically related derivatives thereof, or its monosodium phosphate derivative; deoxycholic acid, pharmaceutically acceptable salts thereof, pharmaceutically acceptable metabolically related derivatives thereof, or its monosodium phosphate derivative; diacetyl tartaric acid esters of (M)mono- and diglycerides or their monosodium phosphate derivatives, glycocholic acid, pharmaceutically acceptable salts thereof, pharmaceutically acceptable metabolically related derivatives thereof, or its monosodium phosphate derivative, mono- and diglycerides or their monosodium phosphate derivatives, and combinations thereof.

In a further embodiment, the absorption enhancer comprises about 500 millimolar bile acids, pharmaceutically acceptable salts thereof, or pharmaceutically acceptable metabolically related derivatives thereof. In a further embodiment, the absorption enhancer is lipophilic and carries a negative charge at mammalian physiological pH ranges. In a further embodiment, the absorption enhancer concentration is about 500 millimolar. In a further embodiment, a method is disclosed for applying a bethanechol, absorption enhancer, liquid carrier composition to the posterior pharynx.

In a further embodiment, the application is spraying, swabbing, squirting, or gargling. In a further embodiment, the muscarinic receptor agonist is bethanechol; the application is spraying and each spray delivers greater than about 0.1 ml but less than about 0.5 ml; and the concentration of bethanechol is between about 3 mg/ml and about 15 mg/ml. In a further embodiment, the concentration of bethanechol is about 5 mg/ml.

In another general aspect, a method of treating a patient suffering from ineffective esophageal motility is described. The method comprises topically applying a therapeutically effective amount to the back of the esophagus of the patient, a composition comprising from about 15 millimolar to about 700 millimolar bethanechol or pharmaceutically acceptable salts thereof; about 500 millimolar one or more pharmaceutically acceptable bile acids; and a pharmaceutically acceptable liquid carrier; and wherein parasympathetic nervous system side effects normally associated with bethanechol are reduced or virtually eliminated.

In a further embodiment, the therapeutically effective amount is between about 3 mg and about 15 mg. In another general aspect, a method is disclosed for facilitating a therapeutic esophageal peristaltic effect to a patient in need of treatment while reducing or virtually eliminating a parasympathetic nervous system side effect. The method comprises topically administering to the esophagus a therapeutically effective amount of bethanechol to facilitate esophageal peristalsis so that the parasympathetic nervous system side effect is reduced while maintaining the therapeutic esophageal peristaltic effect.

In a further embodiment, the therapeutically effective amount of bethanechol is between about 3 mg and about 10 mg. In a further embodiment, the patient in need suffers from acquired immune deficiency syndrome, stroke, amyotrophic lateral sclerosis, acid reflux disease, spinal cord injury, Parkinson's Disease, Alzheimer's Disease, multiple sclerosis, and combinations thereof.

In a further embodiment, a method for improving the absorption of a topical pharmaceutical treatment possessing an active ingredient with a stable ionic charge at physiological pH is described. The method includes the following steps: combining the active ingredient with a complimentary counter-ionic molecule, the counter-ionic molecule comprising a charged moiety for complimenting the charge of the active ingredient and a hydrophobic moiety; and formulating the treatment for application to the gastrointestinal tract.

Other exemplary embodiments of the invention will become apparent from the detailed description provided hereinafter. It should be understood that the detailed description and specific examples, while disclosing exemplary embodiments of the invention, are intended for purposes of illustration only and are not intended to limit the scope of the invention.

### DETAILED DESCRIPTION

Bethanechol is known to be poorly absorbed by the mucosal lining of the gastrointestinal tract. It is a stable, water-soluble molecule, but has a positive charge on a quaternary amine which hampers its absorption. Thus, to achieve the beneficial effects of bethanechol on esophageal motility, systemic dosages in the range of 25-50 mg are required. Further, absorption of bethanechol at these dosage levels by the stomach and lower GI tract results in systemic distribution to other areas of the body such as the urinary tract and colon. A 50 mg oral dosage, for example, is believed to be about 22 percent absorbed in the stomach and intestines, which are rich in blood supply. This would result in a systemic level of about 11 mg of bethanechol. As described herein, however, bethanechol delivered locally to the pharynx and esophagus in a form that can more easily be absorbed by the mucosal lining of the pharynx and esophagus and delivered to the muscarinic receptors, principally the M2 receptors, can provide esophageal motility benefits, as well as increased salivation, while minimizing or avoiding systemic absorption and reducing or eliminating undesirable side effects. Enhancing the reduction or elimination of undesirable side effects is the fact that the esophagus has low vascularization, thus inhibiting the systemic absorption of bethanechol.

As used herein, by “pharmaceutically acceptable” it is meant the named ingredient must be compatible with the other ingredients of the formulation, and not deleterious to the recipient thereof.

As used herein, by “therapeutically effective amount” it is meant an amount which provides a therapeutic benefit in the treatment, management, or prevention of conditions that are responsive to the named compound. A pharmaceutically acceptable absorption enhancer can help provide a mechanism whereby the bethanechol is delivered to the esophageal mucosa M2 receptors and to the M2 receptors in the underlying deeper smooth and striated muscles. Included are those absorption enhancers which are lipophilic and carry a negative charge at mammalian physiological pH ranges (5-8). A member of the bile acid group, of which taurocholic acid is exemplary, allows for enhanced absorption of bethanechol by forming an ionic bond with bethanechol at the negative charge of the bile acid (e.g., the sulfate group of taurocholic acid), thereby carrying bethanechol chloride with it. It is also believed that a member of the bile acid group, of which taurocholic acid is exemplary, acts upon the M3 receptors to alleviate or ameliorate the swallowing dysfunction which is often associated with inflammation. In addition to taurocholic acid, bile acids include, but are not limited to, cholic acid, desoxycholic acid, glycocholic acid, and ox bile extract. Special Bile Acid Mix (New Zealand Pharmaceuticals Ltd., Palmerston North, North Island, NZ) has, for example, the following composition:

| | |
|---|---|
| taurocholic acid | 35.8 wt. percent |
| glycocholic acid | 24.8 wt. percent |
| taurochenodeoxycholic acid | 2.0 wt. percent |
| taurodeoxycholic acid | 9.5 wt. percent |
| glycochenodeoxycholic acid | 1.3 wt. percent |
| glycodeoxycholic acid | 5.0 wt. percent |

Other agents include diacetyl tartaric esters of (M)mono- and diglycerides; mono- and diglycerides; monosodium phosphate derivatives of cholic acid; desoxycholic acid; diacetyl tartaric acid esters of (M)mono- and diglycerides, glycocholic acid, and mono- and diglycerides; and propylene glycol.

Without attempting to be all-inclusive, absorption enhancers may be chosen from the following group: ox bile; taurocholic acid, pharmaceutically acceptable salts thereof, or pharmaceutically acceptable metabolically related derivatives thereof; glycocholic acid, pharmaceutically acceptable salts thereof, or pharmaceutically acceptable metabolically related derivatives thereof; glycocholic acid, pharmaceutically acceptable salts thereof, or pharmaceutically acceptable metabolically related derivatives thereof; taurochenodeoxycholic acid, pharmaceutically acceptable salts, or pharmaceutically acceptable metabolically related derivatives thereof; taurodeoxycholic acid, pharmaceutically acceptable salts thereof, or pharmaceutically acceptable metabolically related derivatives thereof; glycochenodeoxycholic acid, pharmaceutically acceptable salts thereof, or pharmaceutically acceptable metabolically related derivatives thereof; ox bile extract; propylene glycol; cholic acid, pharmaceutically acceptable salts thereof, pharmaceutically acceptable metabolically related derivatives thereof, or its monosodium phosphate derivative; deoxycholic acid, pharmaceutically acceptable salts thereof, pharmaceutically acceptable metabolically related derivatives thereof, or its monosodium phosphate derivative; diacetyl tartaric acid esters of (M)mono- and diglycerides or their monosodium phosphate derivatives, glycocholic acid, pharmaceutically acceptable salts thereof, pharmaceutically acceptable metabolically related derivatives thereof, or its monosodium phosphate derivative, mono- and diglycerides or their monosodium phosphate derivatives, and combinations thereof.

Adding pharmaceutically acceptable thickening agents and viscosity enhancers will also increase the viscosity of the formulation which will act to increase adherence to the esophageal mucosa, and, thus increase contact time. This increased contact time, along with the presence of absorption enhancers will facilitate absorption of bethanechol.

Adding pharmaceutically acceptable flavorings, sweetening enhancers, and bitterness suppressors such as those offered by FlavorX, Inc. (Columbia, Md.) can make the formulation more palatable, and, thus, more likely to be used. Pharmaceutically acceptable antioxidants and antimicrobials can also be added.

It is believed that, as a result of activation of the M2 receptors, pressure within the upper esophagus will increase, initiating a peristaltic wave in the upper esophagus moving toward the lower esophageal sphincter (LES). The formulation will continue to flow by gravitational forces and dissolution in saliva to the LES, where M2 receptors in the LES will be activated by bethanechol to increase pressure of the LES, helping to prevent reflux of gastric contents back into the esophagus. An exemplary formulation may be prepared by combining between 360 and 900 mg bethanechol chloride, about 600 mg of sodium carboxymethylcellulose or about 240 mg of methylparaben, about 24 mg of glycerin, an absorption enhancer, and preserved water quantum 20 sufficiat to 120 ml total volume of the formulation. Such formulation is suitable for topical spray application to the pharynx and esophagus.

In one general aspect, the composition is effective for treating a patient suffering from ineffective esophageal motility and comprises from about 15 millimolar to about 70 millimolar bethanechol or pharmaceutically acceptable salts thereof, about 500 millimolar one or more pharmaceutically acceptable bile acids, and a pharmaceutically acceptable liquid carrier.

An exemplary formulation comprises 25 millimolar bethanechol, 500 millimolar Special Bile Acid Mix, and sufficient viscosity enhancers in an aqueous solution. In a further exemplary formulation, the molar concentration of taurocholic acid and taurodeoxycholic acid is present in a ratio of about 10:1 to about 40:1 with the molar concentration of bethanechol. Such formulation is suitable for topical spray application to the pharynx and esophagus. The 40:1 ratio balances the need to have an excess of absorption enhancers relative to the bethanechol with cost. The higher the ratio, the less bethanechol that would be required to achieve similar absorption.

An exemplary method comprises treating ineffective esophageal motility while reducing or virtually eliminating an undesirable side effect, particularly a parasympathetic nervous system side effect, in a mammal in need of treatment by topically administering to the pharynx and esophagus of the mammal a therapeutically effective amount of bethanechol and an absorption enhancer to facilitate esophageal peristalsis so that the undesirable side effect, particularly the parasympathetic nervous system side effect, is reduced while maintaining the therapeutic esophageal peristaltic effect. A further exemplary method comprises delivering an amount of bethanechol of between about 3 mg and about 15 mg. A further exemplary method comprises introducing about ten 0.1 ml sprays comprising 5 mg bethanechol per ml and an absorption enhancer. Of the total 5 mg introduced, it is expected about 25 percent of the bethanechol, or about 1.25 mg, is adsorbed. The remaining 3.75 mg would proceed into the stomach and intestines, and, at about a 22 percent absorption rate would result in only about 0.8 mg bethanechol being systemically adsorbed, far below the threshold necessary to produce undesirable side effects.

In one general aspect, delivery is effected by methods effective to introduce the composition to the back of the throat, pharynx, and/or esophagus such that the composition topically contacts the tissues thereof. Examples include a pump spray, for example, a pump spray comprising an extension tube which facilitates delivery deep into the back of the throat. In another general aspect the composition is squirted as a stream or partial stream into the back of the throat. In a further general aspect, the composition is swabbed into the back of the throat. In a further embodiment, the composition is gargled in the back of the throat.

### EXAMPLES

A formulation was prepared consisting essentially of 600 mg bethanechol chloride; 600 mg sodium carboxymethylcellulose; 24 ml glycerin; and 29.5 g Special Bile Acid Mix, the Special Bile Acid Mix consisting essentially of 35.8 wt. percent taurocholic acid, 24.8 wt. percent glycocholic acid, 2.0 wt. percent taurochenodeoxycholic acid, 9.5 wt. percent taurodeoxycholic acid, 1.3 wt. percent glycochenodeoxycholic acid, and 5.0 wt. percent glycodeoxycholic acid; mixed in preserved water quantum sufficiat 120 ml total volume of formulation.

On three separate occasions, a healthy male volunteer introduced 10 about 0.1 ml sprays of the above formulation to the back of his throat. After about 10 minutes, the volunteer reported easier swallowing and increased salivation. These effects continued for approximately 90 minutes. At no time during the test did the volunteer experience any undesirable side effects and, particularly, no side effects commonly associated with the administration of high systemic doses of bethanechol.

On five separate occasions, a second healthy male volunteer also introduced 10 about 0.1 ml sprays of the above formulation to the back of his throat. As with the first volunteer, after about 10 minutes, the second volunteer also reported easier swallowing and increased salivation that continued for about 90 minutes. Also as with the first volunteer, at no time during the test did the second volunteer experience any undesirable side effects and, particularly, no side effects commonly associated with the administration of high systemic doses of bethanechol. Neither volunteer experienced any long-term effects.

Ultra manometry was performed on 5 patients employing a 5mg/ml formulation. The patients were given ten 5 ml swallows of water and then administered the formulation to the back of the throat. After allowing 10 minutes for absorption, the patients were then given another ten 5 ml swallows. None of the patients experienced undesirable parasympathetic nervous system side effects. The data is summarized in the following table:

| | peri- | nor- | | | | | |
|---|---|---|---|---|---|---|---|
| | staltic | mal | Hypo | Failed | Simultan | Amp 3 | Amp 7 |
| | % | % | % | % | % | mmHG | mmHg |
| 1 pre-test | 0 | 0 | 0 | 94 | 6 | 31.9 | 29.6 |
| 2 pre-test | 10 | 0 | 10 | 10 | 80 | 30.7 | 31.1 |
| 3 pre-test | 70 | 30 | 40 | 30 | 0 | 49.3 | 53.4 |
| 4 pre-test | 30 | 0 | 30 | 60 | 10 | 23.1 | 34.8 |
| 5 pre-test | 30 | 0 | 30 | 70 | 0 | 29.4 | 16.5 |
| 1 post-test | 57 | 0 | 57 | 36 | 7 | 77.7 | 55.6 |
| 2 post-test | 69 | 0 | 69 | 31 | 0 | 26.9 | 29.1 |
| 3 post-test | 90 | 10 | 80 | 0 | 10 | 46.4 | 43.4 |
| 4 post-test | 70 | 0 | 70 | 30 | 0 | 35.2 | 30.7 |
| 5 post-test | 50 | 0 | 50 | 50 | 0 | 32.1 | 12.9 |

| | Amp 11 | mean 3/7 | | CFV | Vel 11-3 |
|---|---|---|---|---|---|
| | mmHg | mmHg | DCI | cm/sec | cm/s |
| 1 pre-test | 29.1 | 30.8 | 106.4 | | |
| 2 pre-test | 27.8 | 31 | 129.5 | 5.72 | 19.3 |
| 3 pre-test | 49.9 | 51.7 | 302.2 | 2 | 2.5 |
| 4 pre-test | 25.3 | 28.9 | 225.1 | 4.1 | 4 |
| 5 pre-test | 27.3 | 22.9 | 128.1 | 3 | 4.1 |
| 1 post-test | 35.5 | | 622.4 | 4.8 | 6.4 |
| 2 post-test | 30.8 | 28 | 85.1 | 3.7 | 3.4 |
| 3 post-test | 19.1 | 44.9 | 256.6 | 2.9 | 2.6 |
| 4 post-test | 26 | 32.9 | 297.5 | 2.9 | 3.8 |
| 5 post-test | 22.7 | 22.5 | 100 | 2.4 | 2.3 |

A formulation was prepared comprising 1200 mg bethanechol chloride; 600 mg sodium carboxymethylcellulose; 24 ml glycerine; and 36.9 g Special Bile Acid Mix; mixed in preserved water quantum sufficiat 120 ml total volume of formulation.

Having shown and described an embodiment of the invention, those skilled in the art will realize that many variations and modifications may be made to affect the described invention and still be within the scope of the claimed invention. Additionally, many of the elements indicated above may be altered or replaced by different elements which will provide the same result and fall within the spirit of the claimed invention. It is the intention, therefore, to limit the invention only as indicated by the scope of the claims.

One extension of this work into treatment of GERD is found from a publication entitled “A Missing Sphinteric Component of the Gastro-Esophageal Junction in Patients with GERD” by Miller, et al, Neurogastroenterol Motil. 2009 August; 21(8): 813-e52. Doi:10.1111/j.1365-2982.2009.01294.x, which is incorporated by reference as if fully recited herein.

Another extension of this work leads to development of a method for effecting the absorption of a topical pharmaceutical treatment that has an active ingredient with a stable ionic charge at physiological pH. The method is based upon principles of partition coefficient. In such a method, the active ingredient to be absorbed is selected and the stable ionic charge at physiological pH is determined. The active ingredient selected preferably has a weight of 500 dalton or less.

An agent for enhancing absorption of the active ingredient is then selected. An appropriate agent will likely be a complimentary counter-ionic molecule that comprises a hydrophobic moiety and a charged moiety that compliments the charge of the active ingredient.

More than one absorption-enhancing agent may qualify under this criterion. To determine how to optimize the agent for the active ingredient, a test is conducted. A hydrophobic and a hydrophilic vehicle are selected for the test. An exemplary hydrophobic vehicle is n-octanol and an exemplary hydrophilic vehicle is water. An amount of the active ingredient is placed in the hydrophilic vehicle at a known concentration. An amount of the hydrophobic vehicle is added to the hydrophilic vehicle and the resulting mixture is shaken for a period of time, effecting extraction of the active ingredient into the hydrophobic vehicle. By measuring the concentration of the active ingredient in one of to vehicles, the base level partition coefficient is determined.

At least one proposed absorption-enhancing agent is then selected and the liquid-liquid extraction is repeated at least twice, using the same hydrophilic and hydrophobic agents. By changing the amount of the absorption-enhancing agent and determining the partition coefficient in each test, the ability of the selected agent to enhance absorption in vivo rather than in vitro is determined.

This detailed description is intended principally as a description of the present embodiments of the invention, and is not intended to represent the only form in which the present invention may be synthesized, formed, or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

What is claimed is:

1. A method for effecting the gastrointestinal absorption of a pharmaceutical treatment that has an active ingredient with a stable ionic charge at physiological pH comprising:
combining the active ingredient with a complimentary counter-ionic molecule, the counter-ionic molecule comprising a charged moiety for complimenting the charge of the active ingredient and a hydrophobic moiety; and
formulating the treatment for application to the gastrointestinal tract.

2. The method of claim 1, wherein:
the active ingredient is bethanechol.

3. The method of claim 2, wherein:
the counter-ionic molecule comprises one or more bile acids.

4. The method of claim 3, wherein:
the molar ratio of counter-ionic molecule to active ingredient is at least 10:1.

5. The method of claim 4, wherein:
the bile acids comprise taurocholic acid, glycocholic acid, taurochenodeoxycholic acid, taurodeoxycholic acid, glycochenodeoxycholic acid, and glycodeoxycholic acid.

6. The method of claim 5, wherein:
the bile acids are present in approximately the following amounts: taurocholic acid from about 30 to about 40 wt. percent; glocoycholic acid from about 20 to about 30 wt. percent; taurochenodeoxycholic acid from about 1 to about 5 wt. percent; taurodeoxycholic acid from about 5 to about 10 wt. percent; glycochenodeoxycholic acid from about 1 to about 5 wt. percent; and glycodeoxycholic acid from about 1 to about 10 wt. percent.

7. A topical pharmaceutical composition for the treatment of ineffective esophageal motility, comprising:
from about 15 millimolar to about 70 millimolar bethanechol or pharmaceutically acceptable salts thereof;
about 500 millimolar one or more pharmaceutically acceptable bile acids; and
a pharmaceutically acceptable liquid carrier, wherein parasympathetic nervous system side effects normally associated with the administration of bethanechol are reduced or virtually eliminated.

8. The composition of claim 7, wherein:
the concentration of bethanechol is about 25 millimolar.

9. The composition of claim 8, wherein:
the bile acids comprise:
taurocholic acid, pharmaceutically acceptable salts thereof, or pharmaceutically acceptable metabolically related derivatives thereof; taurochenodeoxycholic acid, pharmaceutically acceptable salts thereof, or pharmaceutically acceptable metabolically related derivatives thereof; and taurodeoxycholic acid, pharmaceutically acceptable salts thereof, or pharmaceutically acceptable metabolically related derivatives thereof.

10. The composition of claim 9, wherein:
the pharmaceutically acceptable liquid carrier is water.

11. A method for treating ineffective motility of the esophagus in a patient, comprising the step of:
administering, in a therapeutically effective amount, a composition comprising:
from about 15 millimolar to about 60 millimolar bethanechol or pharmaceutically acceptable salts thereof;
about 500 millimolar total concentration of one or more pharmaceutically acceptable bile acids; and
a pharmaceutically acceptable liquid carrier;
wherein parasympathetic nervous system side effects normally associated with bethanechol are reduced or virtually eliminated.

12. The method of claim 11, wherein:
the administering step is achieved by applying the composition topically to the back of the esophagus.

13. The method of claim 11, wherein:
the administering step is achieved by applying the composition intramuscularly.

14. The method of claim 11, wherein:
the therapeutically effective amount is between about 3 mg and about 7.5 mg.

15. The method of claim 11, wherein:
the therapeutically effective amount is sufficient to close the lower esophageal sphincter of the patient.

16. A method for treating gastroesophageal reflux disease (GERD) in a patient, comprising the steps of:
topically applying a composition, in a therapeutically effective amount, to the back of the esophagus, the composition comprising:
from about 15 millimolar to about 60 millimolar bethanechol or pharmaceutically acceptable salts thereof;
about 500 millimolar total concentration of one or more pharmaceutically acceptable bile acids; and
a pharmaceutically acceptable liquid carrier;
wherein parasympathetic nervous system side effects normally associated with bethanechol are reduced or virtually eliminated.

17. A method for effecting the topical absorption of a pharmaceutical treatment having an active ingredient that has a stable ionic charge at physiological pH, the method comprising the steps of:
selecting at least one complimentary counter-ionic molecule as an absorption enhancing agent for the active ingredient, each complimentary counter-ionic molecule comprising a hydrophobic moiety and a and a charged moiety that compliments the charge of the active ingredient;
combining the active ingredient with the absorption-enhancing agent; and
formulating the treatment for topical application.

18. The method of claim 17, wherein:
the active ingredient has a molecular weight less than 500 daltons.

19. The method of claim 18, wherein:
the active ingredient is water soluble.

20. The method of claim 18, wherein:
the step of selecting the absorption-enhancing agent comprises the steps of:
establishing a base line partition coefficient of the active ingredient by the steps of:
adding a predetermined amount of the active ingredient to a hydrophilic vehicle;
conducting a liquid-liquid extraction of the active ingredient into a hydrophobic vehicle; and
measuring an amount of the active ingredient that has been extracted by the hydrophobic vehicle;
establishing at least one partition coefficient for the active ingredient in combination with at least one amount of each of the at least one complimentary counter-ionic molecules, each partition coefficient determined by the steps of:
adding the predetermined amount of the active ingredient and a predetermined amount of the complimentary counter-ionic molecule to the hydrophilic vehicle;
conducting a liquid-liquid extraction of the active ingredient into a hydrophobic vehicle; and
measuring an amount of the active ingredient that has been extracted by the hydrophobic vehicle; and
selecting the identity and amount of the absorption-enhancing agent based upon the partition coefficients established.
